(19) United States
(12) Patent Application Publication
(10) Pub. No.: US 2002/0034524 A1
(43) Pub. Date: Mar. 21, 2002

(54) Cosmetic compositions containing keratinization modulators and methods for improving keratinous surfaces

(76) Inventor: Jacques Louis Poret, Sevran (FR)

Correspondence Address: Julie Blackburn, Revlon Consumer Products Corporation, 625 Madison Avenue, New York, NY, 10022, US

(21) Appl. No.: 09/879,708

(22) Filed: Jun. 12, 2001

Related U.S. Application Data
(60) Provisional application No. 60/212,269, filed on Jun. 19, 2000.

(51) Int. Cl. A61K 7/06; A61K 31/404

(52) U.S. Cl. 424/401; 514/419

(57) ABSTRACT

A method for cosmetically treating human keratinous surfaces to ameliorate the adverse effects of aging and environment thereon by modulating keratinization of superficial epithelial cells forming said keratinous surfaces, comprising contacting said keratinous surfaces with a lipophilic keratinization modulating compound in a cosmetically acceptable carrier; a method for modulating keratinization of superficial epithelial cells, and a cosmetic composition for application to human keratinous surfaces comprising a receptor negative, lipophilic methoxytryptamine compound in a cosmetically acceptable carrier.

### RELATED APPLICATIONS

This application claims priority from provisional patent application Ser. No. 60/212,269, filed Jun. 19, 2000.

### TECHNICAL FIELD

The invention is in the field of cosmetic compositions for application to human keratinous surfaces for the purpose of ameliorating the adverse effects of aging and the environment thereon.

### BACKGROUND OF THE INVENTION

The skin of youth tends to be dewy, fresh, and radiant. This may be due in part to the relatively rapid cell turnover rate in young skin. Cell turnover rate is defined as the amount of time it takes for an epithelial cell created in the basal cell layer to migrate to the top epidermal layer, or stratum corneum, and be sloughed off.

Skin has five layers. The innermost basal layer, or stratum basale, is where epithelial cells are created via mitosis. The next innermost layer is the stratum spinosum, or spiny layer, which contains prominent intercellular bridges that connect adjacent cells. The cells in the stratum spinosum have high levels of RNA, which is needed for protein synthesis and keratin production. The stratum granulosum, or granular layer, is where cells are filled with keratin and moved to the skin's surface—a process referred to a keratinization. Keratin is a highly fibrous protein that, when incorporated into cells, causes cell walls to harden. The stratum lucidum is a layer that is very prominent in thicker skin areas such as the palms of hands and soles of the feet. Finally, the stratum corneum is the outermost layer of skin and is composed of sebum and thin, dead, platelet like epithelial cells that are continuously shed and replaced.

Generally, the cell turnover rate in young skin is about 3 weeks which means that it takes about 3 weeks for an epithelial cell created in the basal layer to migrate to the stratum corneum and be sloughed off. Because the entire process takes a relatively short time in young skin, the keratin containing cells, or keratinocytes, are sloughed from the skin before the keratin has an opportunity to further cross-link and become even more rigid. This is why the keratin found in young skin is mostly “new” or “soft” keratin, which contributes to the luminosity and radiance often found in that type of skin. As skin ages, the cell turnover rate slows significantly, reaching 4 to 5 weeks in middle age. Keratinocytes that remain in the skin longer tend to become more keratinized, i.e. the keratin in the cells cross-links and causes the formation of a more rigid, “aged” cell. Accordingly, aged skin contains a mixture of hard and soft keratin, and the hard or aged keratin will provide certain, less than desirable aesthetic properties to the skin. In addition, the reduced cell turnover rate in combination with environmental assaults and other systemic changes leads to other properties found in aging skin such as wrinkling, sagging, dryness, and sallowness.

Many of the current skin treatment products are designed to improve skin appearance by sloughing off the top layers of the epidermis. This, in turn, promotes more rapid cell turnover as the skin works overtime to make new epithelial cells to ensure that the skin maintains a constant thickness. Alpha hydroxy acids are an example of skin treatment additives that act by sloughing off the top layers of the epidermal keratinocytes. A similar effect can be achieved with mechanical exfoliation of the skin with loofahs and abrasive scrubs. One problem with this approach is that the constant cycle of epidermal sloughing and increased cell turnover may cause skin to become too sensitive, which may result in dryness, chapping, and other problems.

The ideal cosmetic additive for skin care products will not promote more rapid cell turnover or excessive keratinization, but rather will modulate keratinization so that keratinocytes that remain on the skin surface for longer periods of time do not contain excessive amounts of keratin, or keratin that is excessively hard or cross-linked. This desirable additive will have no systemic, or pharmacological, effect on the body when it is applied to the keratinous surface, and preferably will not penetrate the epidermis.

It has been discovered that certain compounds that are capable of modulating keratinization in human epithelial cells exhibit beneficial properties when applied to human keratinous surfaces such as skin, nails, or hair. When such compounds are incorporated into cosmetic compositions, and applied to human keratinous surfaces, the keratinous surfaces exhibit improvements in texture, radiance, luminosity, and other aesthetic attributes.

It is an object of the invention to provide a method for cosmetically treating keratinous surfaces to ameliorate the adverse effects of aging and the environment by treating the keratinous surface with a cosmetic composition containing a lipophilic keratinization modulating compound that is, preferably, receptor inactive.

It is a further object of the invention to provide a method for modulating keratinization in human superficial epithelial cells forming human keratinous surfaces, comprising applying to said keratinous surfaces a cosmetic composition comprising a lipophilic, keratinization modulating compound in a cosmetically acceptable carrier.

It is a further object of the invention to provide a method for treating keratinous surfaces to ameliorate the adverse effects of aging and the environment, by contacting the keratinous surface with a keratinization modulating compound that does not have a systemic effect on the body.

It is a further object of the invention to provide a method for treating keratinous surfaces to ameliorate the adverse effects of aging and the environment with a keratinization modulating compound that is topically delivered to the keratinous surface in the form of a cosmetic composition.

It is a further object of the invention to provide cosmetic compositions for treating keratinous surfaces, comprising an effective amount of a keratinization modulating compound in a cosmetically acceptable carrier.

### SUMMARY OF THE INVENTION

The invention comprises a method for cosmetically treating human keratinous surfaces to ameliorate the adverse effects of aging and environment thereon by modulating keratinization of superficial epithelial cells forming said keratinous surfaces, comprising contacting said keratinous surfaces with a lipophilic keratinization modulating compound in a cosmetically acceptable carrier.

The invention further comprises a method for modulating keratinization in human superficial epithelial cells forming human keratinous surfaces, comprising applying to said keratinous surfaces a cosmetic composition comprising a lipophilic, keratinization modulating compound in a cosmetically acceptable carrier.

The invention also comprises a cosmetic composition for application to human keratinous surfaces comprising a receptor negative, lipophilic methoxytryptamine compound in a cosmetically acceptable carrier.

### DETAILED DESCRIPTION

All percentages mentioned herein are percentages by weight unless otherwise indicated.

The phrase “ameliorate the adverse effects of aging” means to superficially improve skin conditions often seen in aging skin, such as dryness, uneven pigmentation, rough texture, laxity, wrinkling, sallowness, lack of clarity, and so on.

The phrase “ameliorate the adverse effects of environment” means to superficially improve skin conditions often seen in skin that is exposed to environmental assaults such as sun, wind, cold, heat, pollution, and the like, and as a result may exhibit conditions such as sunburn, windburn, chapping, rash, clogged pores, blackheads, whiteheads, or acne vulgaris.

The term “human superficial epithelial cells” means epithelial cells found on human body (keratinous) surfaces such as skin, nails, or hair.

The term “keratinization modulating compound” means a compound that is capable of affecting keratinization of epithelial cells, either by beneficially impacting the amount of keratin incorporated into the epithelial cells during the keratinization process, or by exerting a beneficial effect on the keratin that is already present in human superficial epithelial cells. A keratinization modulating compound will generally either reduce the amount of keratin incorporated into epithelial cells, or cause the keratin already present in human superficial epithelial cells to be less “hard” or “rigid”, e.g. “aged”. Similarly, the term “modulating keratinization” means beneficially regulating the amount of keratin incorporated into cells, or the effects of keratinization on the superficial epithelial cells of human keratinous surfaces.

The term “lipophilic” means that the keratinization modulating compound is soluble or dispersible in nonpolar materials such as waxes and oils.

The term “receptor inactive” means that the compound does not stimulate receptors for fibroblast growth factors, vascular endothelial growth factors, macrophage colony stimulating factors, or transmembrane 4 proteins.

I. THE METHOD

The invention first comprises a method for cosmetically treating human keratinous surfaces to ameliorate the adverse effects of aging and environment thereon by modulating keratinization of superficial epithelial cells forming said keratinous surfaces, comprising contacting said keratinous surfaces with a lipophilic keratinization modulating compound in a cosmetically acceptable carrier.

The invention further comprises a method for modulating keratinization in human superficial epithelial cells forming human keratinous surfaces, comprising applying to said keratinous surfaces a cosmetic composition comprising a lipophilic, keratinization modulating compound in a cosmetically acceptable carrier.

In the method of the invention, the lipophilic kertinization modulating compound is applied to the keratinous surface in the form of a cosmetic composition. A wide variety of cosmetic compositions are suitable as the vehicle, provided that the keratinization modulating compound is stable therein, and adequately solubilized or dispersed in the cosmetic composition such that when the composition is applied to the desired keratinous surface, the keratinization modulating compound is bioavailable thereon, and capable of exerting its beneficial effects. Generally, the amount of keratinization modulating compound found in the cosmetic composition ranges from about 0.001-30%, preferably 0.005-25%, more preferably 0.01-20% by weight of the total composition.

The keratinization modulating compound is, preferably, a methoxytryptamine compound. More preferably, the methoxytryptamine compound is substituted with one or more lipophilic moieties that confer lipophilicity to the compound. Particularly preferred is a compound of the formula:

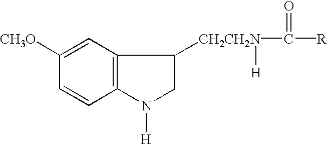

wherein R is a C11 to 19 linear or branched, saturated or unsaturated, alkyl, that may be substituted with one or more hydroxyl groups. Most preferred is a compound where R is C15 and the compound is palmitoyl methoxytryptamine.

The keratinization modulating compound, in a cosmetically acceptable carrier, may be applied to a wide variety of keratinous surfaces such as skin, hair, and nails.

A. Treatment of Skin

In the case where the keratinous surface is skin, the cosmetic composition containing the keratinization modulating compound is preferably applied to the keratinous surface from one to ten times per day in the form of face or body washes, scrubs, toners, serums, skin lotions, or skin creams. In the case where facial skin is being treated, it may be desired to apply color cosmetics containing the keratinization modulating compound. For example, the user may first wash the face with a skin cleansing lotion or cream to remove dirt and excess sebum. Thereafter, the skin is cleansed using a toner that also contains the keratinization modulating compound. Then, a skin lotion or cream containing the compound may be applied to the skin, followed by foundation, eyeshadow, blush, mascara, and powder. In each case, each cosmetic composition may contain the keratinization modulating compound in the appropriate amount. Generally, when all of the products used by the consumer contain the keratinization modulating compound, it may be desirable to use less of the compound in each composition. However, when the user utilizes only one item in the series, e.g. the face cream, it may be desirable to incorporate more of the keratinization modulating compound into the item that is most frequently used by the consumer. If it is known that some consumers use only certain portions of the skin care line, it may be desirable to incorporate larger amounts of the keratinization modulating compound into the products that are most frequently used by consumers, such as the skin cream or lotion. Suggested ranges of keratinization modulating compound incorporated into skin creams or lotions are about 0.1-15%, preferably 0.5-10% by weight of the total composition.

B. Nail Treatment

In the case where the keratinous surface is nails, it is desirable to incorporate the keratinization modulating compound into nail enamels, nail treatment products such as nail hardeners, cuticle creams, and the like. Use of the keratinization modulating compound on nails will cause softening, or plasticization, of the nail surface resulting in loss of brittleness. Brittleness, in turn, is one of the major contributors to nail breakage. The amount of keratinization modulating compound used in the various nail products mentioned is as mentioned above. Suitable amounts of keratinization modulating compound for use in nail care compositions ranges from about 0.001-20%, preferably 0.005-15%, more preferably 0.01-10% by weight of the total composition.

C. Hair

The keratinization modulating compound may be incorporated into one or more cosmetic compositions which are used to treat hair, such as shampoos, conditioners, hair sprays, gels, mousses, and the like. Generally, the keratinization modulating compound will cause the hair to become softer and more manageable, and contribute to enhanced shininess. Suitable ranges of the keratinization modulating compound in hair products ranges from 0.001-20%, preferably 0.005-15%, more preferably 0.01-10% by weight of the total composition.

When incorporated into the cosmetic compositions, the keratinization modulating compound is capable of exerting a variety of beneficial effects on the keratinous surface. In the case of skin, the skin becomes smoother, softer, more toned, and the appearance of wrinkles and lines are reduced. In the case of hair, the hair is shinier and softer. In the case of nails, the nails are less dry and brittle.

II. COMPOSITIONS

The cosmetic compositions used in the method of the invention are further described below.

In addition, the invention further comprises a cosmetic composition for application to human keratinous surfaces comprising a receptor negative, lipophilic methoxytryptamine compound in a cosmetically acceptable carrier.

A. Skin Treatment Compositions

1. Skin Lotions and Creams

The keratinization modulating compound may be incorporated into skin lotions and creams, which are an excellent delivery vehicle for the compound. Lotions and creams may be in the form of water-in-oil or oil-in-water emulsions, or anhydrous creams. Preferably, the lotions and creams are in the form of emulsions, in particular water-in-oil emulsions. Typical emulsions contain about 1-95%, preferably 5-90%, more preferably 10-85% water and about 1-95%, preferably 5-90%, more preferably 10-85% oil, in addition to other desirable ingredients such as surfactants, film formers, humectants, preservatives and the like.

(a) Synthetic Polymers

It is desirable that the cosmetic compositions contain one or more synthetic polymers that may act as film formers, surfactants, lubricating oils, and the like. Suitable synthetic polymers include silicone based polymers as well as polymers made from ethylenically unsaturated monomers.

(i) Silicones

Suitable silicones include or nonvolatile silicones. The term “volatile” means that the silicone has a measurable vapor pressure, or a vapor pressure of at least 2 mm of mercury at 20° C. The term “nonvolatile” means that the oil has a vapor pressure of less than 2 mm of mercury at 20° C. Preferred are compositions containing 0.01-65%, preferably 0.05-50%, more preferably 0.1-45% by weight of the total composition of one or more silicone polymers.

Suitable volatile silicones are liquids and generally have a viscosity of 0.5 to 10 centistokes at 25° C. They include linear and cyclic silicones, or mixtures thereof.

Cyclic silicones (or cyclomethicones) are of the general formula:

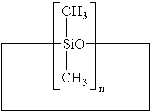

where n=3-7.

Linear volatile silicones in accordance with the invention have the general formula:

(CH₃)₃Si—O—[Si(CH₃)₂—O]ₙ—Si(CH₃)₃

where n=0-6, preferably 0-5.

Linear and cyclic volatile silicones are available from various commercial sources including Dow Coming Corporation and General Electric. The Dow Coming volatile silicones are sold under the trade names Dow Corning 244, 245, 344, and 200 fluids. These fluids comprise octamethylcyclotetrasiloxane, decamethylcyclopentasiloxane, hexamethyldisiloxane, and mixtures thereof.

Also suitable for use in the compositions are nonvolatile silicones including water insoluble, non-emulsifying, nonvolatile silicone fluids such as polyalkyl siloxanes, polyaryl siloxanes, polyalkylaryl siloxane copolymers, and mixtures thereof The nonvolatile silicones may be liquids or solids, but are preferably liquids. Such silicones have the following general formula:

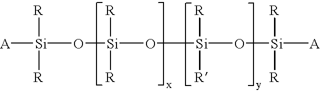

wherein R and R′ are each independently C₁₋₂₂ straight or branched chain alkyl, aryl, alkylamino, alkylaminoalkylamino, and x and y are each independently 0-100,000 with the proviso that there is at least one of either x or y, and A is siloxy endcap unit. Preferred is where A is methyl, and R and R′ are methyl.

Also suitable for use in the compositions are silicone surfactants, which may be nonionic or cationic surfactants. Examples of suitable nonionic silicone surfactants may be liquids or solids at room temperature, and are silicone polymers that have at least one hydrophilic radical and at least one lipophilic radical. The silicone surfactant is generally a water-in-oil or oil-in-water type surfactant which is, and has an Hydrophile/Lipophile Balance (HLB) of 2 to 18. Preferably the surfactant is a nonionic surfactant having an HUB of 2 to 12, preferably 2 to 10, most preferably 4 to 6. The HLB of a nonionic surfactant is the balance between the hydrophilic and lipophilic portions of the surfactant and is calculated according to the following formula:

HLB=7+11.7×log M_{W}/Mₒ

where M_{w} is the molecular weight of the hydrophilic group portion and Mo is the molecular weight of the lipophilic group portion. The hydrophilic portion of the surfactant is generally achieved by substitution onto the polymeric backbone of a radical that confers hydrophilic properties to a portion of the molecule. The hydrophilic radical may be substituted on a terminus of the silicone, or on any one or more repeating units of the polymer. In general, the repeating dimethylsiloxy units of modified polydimethylsiloxane emulsifiers are lipophilic in nature due to the methyl groups, and confer lipophilicity to the molecule. In addition, longer chain alkyl radicals, hydroxy-polypropyleneoxy radicals, or other types of lipophilic radicals may be substituted onto the siloxy backbone to confer further lipophilicity and organocompatibility. If the lipophilic portion of the molecule is due in whole or part to a specific radical, this lipophilic radical may be substituted on a terminus of the organosilicone polymer, or on any one or more repeating units of the polymer. It should also be understood that the organosiloxane polymer in accordance with the invention should have at least one hydrophilic portion and one lipophilic portion.

The term “hydrophilic radical” means a radical that, when substituted onto the silicone polymer backbone, confers hydrophilic properties to the substituted portion of the polymer. Examples of radicals that will confer hydrophilicity are hydroxy-polyethyleneoxy, hydroxyl, carboxylates, and mixtures thereof.

The term “lipophilic radical” means an organic radical that, when substituted onto the silicone polymer backbone, confers lipophilic properties to the substituted portion of the polymer. Examples of organic radicals which will conver lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof The C₁₋₄₀ alkyl may be non-interrupted, or interruped by one or more oxygen atoms, a benzene ring, amides, esters, or other functional groups.

The silicone surfactant used in the invention may have any of the following general formulas:

MₓQ_{y}, or

MₓT_{y}, or

MDₓD′_{y}D″_{z}M

wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RR′SiO_{1.5} or RRSiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:

MDₓD′_{y}D″_{z}M

wherein M=RRRSiO_{½}

D and D′=RR′SiO_{2/2}

D″=RRSiO_{2/2}

x, y, and z are each independently 0-1000,

where R is methyl or hydrogen, and R′ is a hydrophilic radical or a lipophilic radical, with the proviso that the compound contains at least one hydrophilic radical and at least one lipophilic radical.

Most preferred is wherein

M=trimethylsiloxy

D=Si[(CH₃)][(CH₂)ₙCH₃]O_{2/2} where n=0-40,

D′=Si [(CH₃)][(CH₂)ₒ—O—PE)]O_{2/2} where PE is (—C₂H₄O)ₐ(—C₃H₆O)_{b}H, o=0-40,

a=1-100 and b=1-100, and

D″=Si(CH₃)₂O_{2/2}

More specifically, suitable silicone surfactants have the formula:

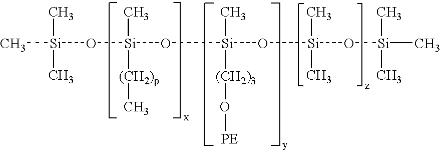

wherein p is 0-40, preferably 12-20, most preferably 15, and

PE is (—C₂H4O)ₐ(—C₃H₆O)_{b}—H

where x, y, z, a, and b are such that the maximum molecular weight of the polymer is approximately 50,000.

Another type of preferred organosiloxane emulsifier suitable for use in the compositions of the invention are emulsifiers sold by Union Carbide under the Silwet™ trademark. These emulsifiers are represented by the following generic formulas:

(Me₃Si)_{y-2}[(OSiMe₂)_{x/y}O—PE]_{y}

wherein PE=—(EO)ₘ(PO)ₙR

R=lower alkyl or hydrogen

Me=methyl

EO is polyethyleneoxy

PO is polypropyleneoxy

m and n are each independently 1-5000

x and y are each independently 0-5000, and

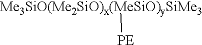

wherein PE=—CH₂CH₂CH2O(EO)ₘ(PO)ₙZ

Z=lower alkyl or hydrogen, and

Me, m, n, x, y, EO and PO are as described above,

with the proviso that the molecule contains a lipophilic portion and a hydrophilic portion. Again, the lipophilic portion can be supplied by a sufficient number of methyl groups on the polymer

Also suitable as nonionic silicone surfactants are hydroxy-substituted silicones such as dimethiconol, which is defined as a dimethyl silicone substituted with terminal hydroxy groups.

Examples of silicone surfactants are those sold by Dow Coming under the tradename Dow Coming 3225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

The preferred skin lotion or cream compositions of the invention contain one or more silicones, preferably selected from a volatile silicone, a nonvolatile silicone fluid, and a silicone surfactant.

(ii) Polymers From Ethylenically Unsaturated Monomers

Also suitable for use in the compositions are one or more synthetic polymers obtained by polymerization of ethylenically unsaturated monomers. Preferred is where the composition contains about 0.01-15%, preferably 0.05-10%, preferably 0.1-8% by weight of the total composition of one or more polymers obtained from ethylenically unsaturated monomers. Examples of suitable polymers are those comprised of ethylenically unsaturated monomers having the general formula:

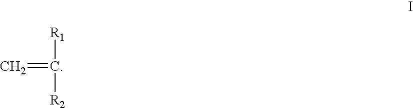

wherein R₁ is H, a C₁₋₃₀ straight or branched chain alkyl, aryl, aralkyl; R₂ is H, CO(NH₂), pyrrolidone, or a substituted or unsubstituted aromatic, alicyclic, or bicyclic ring where the substitutents are C₁₋₃₀ straight or branched chain alkyl, or OCOM or COOM wherein M is H or an alkaline earth or alkali metal salt thereof such as sodium, potassium, calcium, magnesium; a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more hydroxyl groups, or [(CH₂)ₘO]ₙH wherein m is 1-20, and n is 1-200. The polymers may be crosslinked or uncrosslinked. If they are crosslinked, a variety of cross linking agents are suitable, including alcohols, allyl ethers of sucrose, pentaerythritol, or propylene.

Preferably, the polymer is comprised of ethylenically unsaturated monomers of Formula I, above, wherein R₁ is H or a C₁₋₃₀ alkyl, and R₂ is CO(NH₂) or COOM wherein M is H or alkali metal or alkaline earth metal.

More preferably, R₁ is H or CH₃, and R₂ is CO(NH₂) or COOM wherein M is a H or sodium. In the most preferred embodiment of the invention, the composition contains a mixture of two polymers comprised of monomers of Formula I; where in one monomer R₁ is H and R₂ is CO(NH₂); and where in the second monomer R₁ is H, and R₂ is COOM where M is sodium and the polymers are polyacrylamide and sodium polyacrylate.

The preferred compositions contain a mixture of at least one silicone-based synthetic polymer and at least one polymer obtained from ethylenically unsaturated monomers. Particularly preferred are compositions containing a mixture of at least two silicone polymers and at least two polymers obtained from ethylenically unsaturated monomers wherein the silicone polymers are present from 0.1-50% by weight of the total composition and the polymers obtained from ethylenically unsaturated monomers are present in a range of about 0.1-15% by weight of the total composition.

(b) Oils

Preferably, the compositions contain one or more organic oils which are liquid at room temperature. The organic oils may be volatile or nonvolatile. Preferably, the amount of organic oil in the composition ranges from about 0.01-30%, preferably 0.05-25%, more preferably 0.1-20% by weight of the total composition. A variety of organic oils is suitable, including those set forth below:

(i) Paraffinic Hydrocarbons

Suitable oils include are various straight or branched chain paraffinic hydrocarbons having 5 to 60 carbon atoms, more preferably 8-20 carbon atoms. These oils may be volatile or nonvolatile. Suitable hydrocarbons include pentane, hexane, heptane, decane, dodecane, tetradecane, tridecane, and C₈₋₂₀ isoparaffins as disclosed in U.S. Pat. Nos. 3,439,088 and 3,818,105, both of which are hereby incorporated by reference. Preferred volatile paraffinic hydrocarbons have a molecular weight of 70-225, preferably 160 to 190 and a boiling point range of 30 to 320, preferably 60-260° C., and a viscosity of less than 10 centipoise at 25° C. Such paraffinic hydrocarbons are available from EXXON under the ISOPARS trademark, and from the Permethyl Corporation. Suitable C₁₂ isoparaffins are manufactured by Permethyl Corporation under the tradename Permethyl 99A. Another C₁₂ isoparaffin (isododecane) is distributed by Presperse under the tradename Permethyl 99A. Various C₁₆ isoparaffins commercially available, such as isohexadecane (having the tradename Permethyl R), are also suitable.

(ii) Esters

Also suitable are various esters including esters of mono-, di-, and tricarboxylic acids formed by the reaction of mono-, di-, or trifunctional C₁₋₄₀ straight or branched chain alcohols having one or more hydroxyl groups, with C₁-₂₀ straight or branched chain, saturated or unsaturated mono-, di-, or tricarboxylic acids, which may contain one or more hydroxyl groups.

Suitable monoesters have the general formula RCO—OR′ wherein R and R′ are each independently a straight or branched chain, saturated or unsaturated, C₁₋₄₀ alkyl which may be substituted with one or more hydroxyl groups. Preferably, R and R′ are each independently a C₁₋₂₂ straight or branched chain alkyl substituted with one or more hydroxyl groups. Such monoesters are formed by the reaction of a monofunctional alcohol and a carboxylic acid, either of which may be substituted with one or more hydroxyl groups. Examples of suitable monoesters include isononyl isononanoate, coco caprylate/caprate, cetyl lactate, cetyl ricinoleate, stearyl lactate, butyl lactate, decyl oleate, decyl myristate, butyl oleate, glycol paimitate, glycol oleate, glyceryl stearate, glyceryl ricinoleate, and so on.

Suitable diesters include those of the general formula R—CO—Xₙ—CO—R′ wherein R and R′ are each independently a straight or branched chain saturated or unsaturated alkyl which may be substituted with one or more hydroxyl groups, X is —CH₂— or —CHOH—, and n is 0-22. Preferably, in the diester, R and R′ are each independently a C₁₋₂₂ straight or branched chain alkyl which may be substituted with one or more hydroxyl groups, and n is 0-10. Such esters are generally formed by the reaction of a monofunctional alcohol with a dicarboxylic acid, where one or both may be substituted with one or more hydroxyl groups. Examples of such esters include fatty esters of dicarboxylic acids such as adipic, oxalic, succinic, and tartaric acids, including but not limited to dimethyl adipate, dibutyl adipate, dioctyl adipate, distearyl oxalate, dicetyl oxalate, dibutyl succinate, dioctyl succinate, dibutyl tartrate, and so on.

Also suitable are esters of mono-, di-, or trifunctional alcohols and tricarboxylic acids such as citric acid, myristic acid, and so on. Examples of such esters include trioctyldodecyl citrate, tristearyl citrate, trimyristin, triethyl citrate, triacetin, tricaprin, triarachidin, tricapyrlin, and so on.

(c) Natural Polymers

It may also be desirable to include one or more natural polymers in the cosmetic compositions. Suggested ranges of natural polymer are 0.01-15%, preferably 0.05-10%, more preferably 0.1-8% by weight of the total composition. The term “natural polymer” means polymers comprised of naturally occuring monomers, frequently of the carbohydrate variety. Natural polymers may be used exactly as obtained from natural sources, or they may be chemically modified to alter their swelling and solubility characteristics. Examples of natural polymers include biosaccharide gums; aluminum starch octenylsuccinate; chitosan; chitosan derivatives such as chitosan glycolate, chitosan adipate; hyaluronic acid; sodium carboxylmethyl chitin; starch, potassium hyaluronate; and mixtures thereof Particularly preferred as the natural polymer is biosaccharide gum.

(d) Humectants

It may be desirable to include one or more humectants in the compositions. Humectants are hygroscopic materials that retard moisture loss. Suggested ranges of humectant include 0.1-15%, preferably 0.5-10%, more preferably 1-8% by weight of the total composition. Examples of suitable humectants include glycerin; sugars such as maltose, sucrose, xylose; polyethylene glycols; propylene glycol; sodium PCA; allantoin, alkylene glycols, and the like. Particularly preferred are humectants such as glycerine, allantoin, polyethylene glycol, and propylene glycol.

(e) Surfactants

It is desirable that the compositions contain one or more surfactants, especially when the composition is in the emulsion form. Suitable surfactants for use in skin lotions and creams are generally nonionic or cationic surfactants, preferably nonionic surfactants. Preferably, the composition comprises 0.01-20%, preferably 0.05-15%, more preferably 0.1-10% by weight of surfactant.

(i) Nonionic surfactants

Suitable nonionic surfactants are generally compounds produced by the condensation of alkylene oxide groups with a hydrophobic compound. Examples of nonionic surfactants are:

(A) Long chain dialkyl sulfoxides containing one short chain alkyl or hydroxy alkyl radical of from about 1 to 3 carbon atoms and one long hydrophobic chain which may be an alkyl, alkenyl, hydroxyalkyl, or ketoalkyl radical containing from about 8 to 20 carbon atoms, from 0 to 10 ethylene oxide moieties, and 0 or 1 glyceryl moiety.

(B) Polysorbates, such as sucrose esters of fatty acids. Examples of such materials include sucrose cocoate, sucrose behenate, and so on.

(C) Polyethylene oxide condensates of alkyl phenols, for example the condensation products of alkyl phenols having an alkyl group of 6 to 20 carbon atoms with ethylene oxide being present in amounts of about 10 to 60 moles of ethylene oxide per mole of alkyl phenol.

(D) Condensation products of ethylene oxide with the reaction product of propylene oxide and ethylene diamine.

(E) Condensation products of aliphatic alcohols having 8 to 18 carbon atoms with ethylene oxide, for example a coconut alcohol/ethylene oxide condensate having 10 to 30 moles of ethylene oxide per mole of coconut alcohol, the coconut alcohol fraction having 10 to 14 carbon atoms.

(F) Long chain tertiary amine oxides such as those corresponding to the general formula:

R₁R₂R₃NO

wherein R₁ contains an alkyl, alkenyl or monohydroxyalkyl radical ranging from about 8 to 18 carbon atoms in length, from 0 to about 10 ethylene oxide moieties, and from 0 to about 1 glyceryl moiety and R₂ and R₃ are each alkyl or monohydroxyalkyl groups containing from about 1 to about 3 carbon atoms.

(G) Long chain tertiary phosphine oxides corresponding to the general formula:

RR₁R₂PO

wherein R contains an alkyl, alkenyl, or monohydroxyalkyl radical having 8 to 18 carbon atoms, from 0-10 ethylene oxide moieties and 0 or 1 glyceryl moiety, and R₂ and R₃ are each alkyl or monohydroxyalkyl group containing from about 1 to 3 carbon atoms.

(H) Polyethylene glycol (PEG) glyceryl fatty esters, having the formula

RC(O)OCH₂CH(OH)CH₂(OCH₂CH₂)ₙOH

wherein n is 5-200 and RC(O)— is a hydrocarbylcarbonyl group wherein R is preferably an aliphatic radical having 7 to 19 carbon atoms.

(I) C₁₀₋₁₈ alkyl(C₁₋₆)polyhydroxy fatty acid amides such as C₁₂₋₁₈ methylglucamides, N-alkoxy polyhydroxy fatty acid amides, N-propyl through N-hexyl C₁₂₋₁₈ glucamides and so on.

Preferred nonionic surfactants are condensation products of aliphatic alcohols with ethylene oxide. Preferred condensates are formed by the reaction of fatty alcohols and ethylene oxide and include laureth, steareth, beheneth, and the like, having repeating ethylene oxide units ranging from 1 to 100.

(g) Sunscreen

It is particularly desirable to include one or more sunscreens in the compositions. Suitable ranges of sunscreen are 0.01-20%, preferably 0.05-15%, more preferably 0.1-10% by weight of the total composition. A sunscreen is defined as an ingredient that absorbs at least 85 percent of the light in the UV range at wavelengths from 290 to 320 nanometers, but transmits UV light at wavelengths longer than 320 nanometers. Sunscreens generally work in one of two ways. Particulate materials, such as zinc oxide or titanium dioxide, as mentioned above, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorpated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)] amnobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate O, (Octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and so on.

(h) Structuring Agents

It may also be desirable to including one or more structuring agents in the claimed compositions to increase viscosity or otherwise modify the rheology of said compositions. Suitable structuring agents will generally be semi-solid or solid at room temperature and include waxes or wax-like materials such as fatty alcohols, fatty acids, and natural, synthetic, or hydrocarbon waxes. Preferred structuring agents have a melting point of 30 to 120° C., and can be animal waxes, plant waxes, mineral waxes, silicone waxes, synthetic waxes, and petroleum waxes or fatty acids or fatty alcohols. Examples of waxes in accordance with the invention include bayberry, beeswax, candelilla, camauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, PEG-6 beeswax, PEG-8 beeswax, rice bran wax, shellac wax, spent grain wax, sulfurized j ojoba oil, synthetic beeswax, synthetic candelilla wax, synthetic carnauba wax, synthetic japan wax, synthetic jojoba oil, ethylene homo- or copolymers, stearoxy dimethicone, dimethicone behenate, shorea stenoptera butter, stearyl dimethicone, and the like, as well synthetic homo- and copolymer waxes such as PVP/eicosene copolymer, PVP/hexadecene copolymer, and the like.

Also suitable as structuring agents are one or more fatty alcohols having the formula RCH₂OH wherein R is a straight or branched chain saturated or unsaturated alkyl having at least about 6 to 30 carbon atoms. Examples of fatty alcohols suitable for use include behenyl alcohol, C₉₋₁₅ alcohols, caprylic alcohol, cetearyl alcohol, cetyl alcohol, coconut alcohol, decyl alcohol, lauryl alcohol, cetyl alcohol, myristyl alcohol, oleyl alcohol, palm alcohol, stearyl alcohol, tallow alcohol, and the like. The preferred compositions of the invention include a mixture of cetyl and stearyl alcohols.

Preferred compositions comprise one or more structuring agents in the amounts set forth above.

(i) Preservatives

Preferably, the compositions contain one or more preservatives in amounts ranging from 0.001-10%, preferably 0.005-8%, more preferably 0.01-5% by weight of the total composition. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

(j) Vitamins and Antioxidants

The compositions of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof In addition, Vitamins D and K are suitable.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

(k) Protein Derivatives

It may be desirable to incorporate one or more protein derivatives in the compositions. Suitable amounts range from about 0.001-15%, preferably 0.005-10%, more preferably 0.01-8% by weight of the total composition. Protein derivatives are generally prepared by hydrolysis of proteins or reaction of proteins with other chemicals. Generally protein derivatives will provide a film forming and protective effect when used in the compositions. Examples of suitable protein derivatives include hydrolysates of soy, collagen, wheat, casein, albumin, keratin and so on, Also suitable are derivatives of the hydrolysatess such as palmitoyl hydrolyzed wheat protein, stearoyl hydrolyzed keratin, and so on. Preferred are palmitoyl hydrolyzed wheat protein and hydrolyzed soy protein.

2. Hair or Skin Cleansing Compositions

Also suitable as delivery vehicles for the keratinization modulating compound are cosmetic compositions in the form of hair or skin cleansing compositions, which generally comprise about 0.1-35%, preferably 0.5-20%, more preferably 1-15% by weight of the total composition of cleansing surfactant, and 80-99.9%, preferably 85-99.5%, more preferably 90-99% by weight of the total composition of water.

(a) Cleansing Surfactants

Suitable cleansing surfactants may be of the anionic, zwitterionic, nonionic, or betaine type. Suggested ranges of cleansing surfactants are 0.1-25%, preferably 0.5-20%, more preferably 1-15% by weight of the total composition.

(i) Anionic Surfactants

Anionic surfactants include alkyl and alkyl ether sulfates generally having the formula ROSO₃M and RO(C₂H4O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the compositions of the invention are water soluble salts of organic, sulfuric acid reaction products of the general formula:

R₁—SO₃—M

wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, and a sulfonating agent, such as sulfur trioxide.

Also suitable as anionic surfactants are reaction products of fatty acids esterified with isethionic acid and neutralized with sodium hydroxide. The fatty acids may be derived from coconut oil, for example.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid e.g. the dihexyl ester of sodium sulfosuccinic acid, the dioctyl ester of sodium sulfosuccinic acid, and the like.

Other suitable anionic surfactants include olefin sulfonates having about 12 to 24 carbon atoms. The term “olefin sulfonate” means a compound that can be produced by sulfonation of an alpha olefin by means of uncomplexed sulfur trioxide, followed by neutralization of the acid reaction mixture in conditions such that any sultones which have been formed in the reaction are hydrolyzed to give the corresponding hydroxy-alkanesulfonates. The alpha-olefin from which the olefin sulfonate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula:

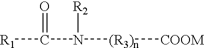

wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms.

(ii) Nonionic Surfactants

Also suitable are certain nonionic surfactants such as alkyl polysaccharides having a hydrophobic group of 6 to 30, preferably 10, carbon atoms and a polysaccharide group such as glucose, galactose, etc. Suitable alkyl polysaccharides are octyl, nonydecyl, undecyldodecyl, tridecyl, tetradecyl, pentadecyl, hexadecyl, heptadecyl, and octadecyl, di-, tri-, tetra-, penta-, and hexaglucosides, galactosides, lactosides, glucoses, fructosides, fructoses, and so on. Particularly preferred is decyl glucoside.

(iii) Amphoteric Surfactants

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate. Suitable amphoteric surfactants may be imidazolinium compounds having the general formula:

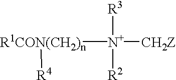

wherein R′ is C₈₋₂₂ alkyl or alkenyl, preferably C₁₂₋₁₆; R² is hydrogen or CH₂CO₂M, R³ is CH₂CH₂OH or CH₂CH₂OCH₂CHCOOM; R⁴ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium cation. Examples of such materials are marketed under the tradename MIRANOL, by Miranol, Inc.

Also suitable amphoteric surfactants are monocarboxylates or dicarboxylates such as cocamphocarboxypropionate, cocoamphocarboxypropionic acid, cocamphocarboxyglycinate, and cocoamphoacetate.

Other types of amphoteric surfactants includ aminoalkanoates of the formula

R—NH(CH₂)ₙCOOM

or iminodialkanoates of the formula:

R—N[(CH₂)ₘCOOM]₂

and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof

(iv) Zwitterionic Surfactants

Zwitterionic surfactants are also suitable for use in the compositions of the invention. The general formula for such surfactants is:

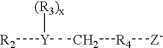

wherein R₂ contains an alkyl, alkenyl or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R⁴ is an alkylene or hydroxyalkylene of from about 1 to about 4 carbon atoms, and Z is a radical selected from the group consisting of carboxylate, sulfonate, sulfate, phosphonate, and phosphate groups.

Zwitterionics include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof Also suitable are sulfo- and amido- betaines such as coco dimethyl sulfopropyl betaine, stearyl dimethyl sulfopropyl betaine, and the like.

Preferred are cleansing compositions for skin, hair, or nails which comprises 1-15% by weight of an anionic surfactant and 0.1-10% by weight of a nonionic surfactant which is an alkyl polysaccharide. Preferred alkylglycosides have the general formula:

R₁—O—(R₂O)ₜ—(G)ₙ—H

wherein R₁ is a linear or branched alkyl or alkenyl radical having 8 to 30 carbon atoms, R₂ is a C₂₋₄ alkylene, (G) is an anhydroglucose unit, t is a number between 0 and 10, preferably 0 to 4, and n is a number from about 1 to 15. Examples of such polyglycosides are sold by Henkel Corporation under the tradenamnes APG 300, APG 350, APG 500, APG 550, APG 625, or the tradename Planteren, e.g. Planteren 300, 600, 1200, 2000, PS-100, and so on. Particularly preferred is decyl glucoside, sold under the tradename Planteren PS 100 by Henkel.

(c) Cationic Conditioning Polymers

The cleansing compositions may contain one or more cationic conditioning polymers which will enhance the beneficial properties of the composition. The cationic polymer may be found in ranges of about 0.1-30%, preferably 0.5-25%, preferably 1-20% by weight of the total composition. Examples of such cationic polymers include:

(i) Quaternary Cellulose Ethers

Quaternary derivatives of cellulose ethers such as polymers sold under the tradename JR-125, JR-400, JR-30M. Preferred is Polyquatermium 10, which is a polymeric quaternary ammonium salt of hydroxyethyl cellulose reacted with a trimethyl ammonium subsituted epoxide.

(ii) Copolymers of Vinylpyrrolidone

Copolymers of vinylpyrrolidone having monomer units of the formula:

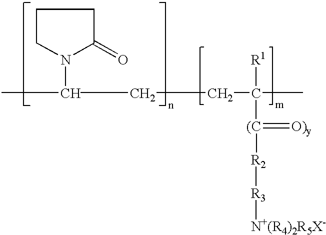

wherein R₁ is hydrogen or methyl, preferably methyl;

y is 0 or 1, preferably 1

R² is O or NH, preferably NH;

R³ is CₓH₂x where x is 2 to 18, or —CH₂—CHOH—CH₂—, preferably CₓH₂ₓ where x is 2;

R⁴ is methyl, ethyl, phenyl, or C₁₋₄ substituted phenyl, preferably methyl; and

R⁵ is methyl or ethyl, preferably methyl.

Preferred are compounds of the above formula wherein y is 1, R² is NH, R³ is CH₂CH₂, R⁴ is methyl, and R⁵ is methyl. Such compounds are known by the CTFA designation Polyquatemium 28.

(iii) Dimethyldiallylammonium Chloride Polymers

Homopolymer of dimethyldiallylammonium chloride, or copolymer of dimethyldiallylammonium chloride and acrylamide. Such compounds are sold under the tradename MERQUAT™ by Merck and are known by the CTFA name Polyquaternium 7.

(iv) Acrylamide Polymers

Homopolymers or copolymers derived from acrylic or methacrylic acid wherein the monomer units are selected from the group consisting of acrylamide, methylacrylamide, diacetone-acrylamide, acrylamide or methacrylamide substituted on the nitrogen by lower alkyl, alkyl esters of acrylic acid and methacrylic acid, vinylpyrrolidone, and vinyl esters. Further examples of cationic polymers that can be used in the compositions of the invention are the cationic polymers disclosed in U.S. Pat. Nos. 5,240,450 and 5,573,709, which are hereby incorporated by reference.

Preferred cleansing composition comprise a mixture of cationic polymers comprising 0.1-10% by weight of Polyquaternium 10 and 0.1-10% by weight of Polyquaternium 7.

3. Nail Enamel or Treatment Compositions

Also suitable for the cosmetic vehicle are nail enamel or nail treatment compositions. Suitable nail enamel compositions generally comprise about 10-95%, preferably 15-85%, more preferably 20-80% by weight of the total composition of solvent, 5-90%, preferably 7-80%, more preferably 8-50% by weight of the total composition of a film forming polymer.

(a) Solvents

The solvent may be aqueous or non-aqueous or a mixture of both types of solvents. Suitable non-aqueous solvents include aliphatic or aromatic ketones such as acetone, diacetone alcohol, dihydroxyacetone, ethyl butyl valerolactone, methyl ethyl ketone, and the like; aliphatic or aromatic alcohols such as methanol, propanol, benzyl alcohol, butoxyethanol, butoxypropanol, butyl alcohol, 3-methyl-3-methoxy-butanol, t-butyl alcohol, butylene glycol, diethylene glycol, abietyl alcohol, propylene carbonate, hexyl alcohol, isopropanol, and the like; glycol ethers; esters such as butyl acetate, ethyl acetate, etc.

(b) Film Forming Polymers

Suitable film forming polymers may be natural polymeric materials such as cellulose or derivatives thereof, or synthetic polymers. Suitable cellulose and cellulose derivative compounds are nitrocellulose, cellulose esters formed by the reaction of cellulose with C₁₋₁₀ carboxylic acids, e.g. cellulose acetate, cellulose butyrate, cellulose acetate isobutyrate, and so on.

Other suitable synthetic polymers include acrylate or methacrylate polymers which are formed from acrylic acid or methacrylic acid esterified with C₁-6 aliphatic alcohols.

(c) Pigments

The nail compositions of the invention may be pigmented or clear. If pigmented, generally 0.1-30% by weight of the total composition, preferably 0.5-20%, more preferably 1-15% of pigment is suggested. Pigments suitable for use in nail enamel compositions are well known and include iron oxides, D&C and FD&C colors, titanium dioxide, and the like. The pigments may be treated or coated with agents which modify the surface properties such as silicones. Examples of silicone treated pigments which can be used in the compositions of the invention are set forth in U.S. Pat. No. 4,832,944, which is hereby incorporated by reference.

4. Hair Conditioning Compositions

Hair conditioning compositions are also a suitable vehicle for the keratinization modulating compound of the invention. Such compositions generally comprise 0.1-30%, preferably 0.5-25%, more preferably 1-20% by weight of the total composition of a cationic surfactant and 0.1-99%, preferably 0.5-95% water.

(a) Cationic Surfactants

Suitable cationic surfactants for use in the hair conditioning compositions include those having the following formula:

Quaternary ammonium salts have the formula:

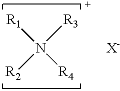

wherein R₁ is an aliphatic group of 1 to 22 carbon atoms, or aromatic, aryl, or alkaryl group having 12 to 22 carbon atoms; R₂ and R₃ are each independently an aliphatic group having 1-22 carbon atoms; and R₄ is an alkyl group of from 1 to 3 carbon atoms, and X is an anion selected from halogen, acetate, phosphate, nitrate and methyl sulfate radicals. The aliphatic groups may contain, in addition to carbon atoms, ether linkages as well as amido groups. Suitable quaternary ammonium compounds may be mono-long chain alkyl, di-long chain alkyl, tri-long chain alkyl, and the like. Examples of such quaternary ammonium salts include behenalkonium chloride, behentrimonium chloride, behentrimonium methosulfate, benzalkonium chloride, benzethonium chloride, benzyl triethyl ammonium chloride, cetalkonium chloride, cetrimonium chloride, cetrimonium bromide, cetrimonium methosulfate, cetrimonium tosylate, cetylpyridinium chloride, dibehenyl/diarachidyl dimonium chloride, dibehenyldimonium chloride, dibehenyldimonium methosulfate, dicapryl/dicaprylyl dimonium chloride,

Other quaternary ammonium salts useful as the cationic conditioning agent are compounds of the general formula:

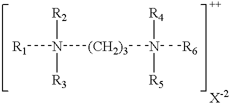

wherein R₁ is an aliphatic group having 16 to 22 carbon atoms, R₂, R₃, R₄, R₅, and R₆ are the same or different and are selected from alkyls having 1 to 4 carbon atoms and X is an anion as above defined.

Amides which exhibit the general formulas set forth below are also suitable conditioning agents:

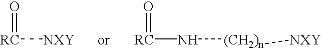

wherein R is a straight or branched chain saturated or unsaturated alkyl having 6 to 30 carbon atoms, n is an integer from 1 to 4, and X and Y are each independently H, or C₁₋₆ lower alkyl.

Preferred is an amide of the formula:

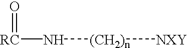

wherein R is a C₁₂₋₂₂ straight or branched chain alkyl, n is an integer from 1 to 4, and X is lower alkyl, preferably methyl.

Also suitable are amidoamine salts, which are the condensation products of fatty acids with a polyfunctional amines, for example, those having the formula RCONH(CH₂)ₙNR₁R₂ where RCO is a fatty acyl group such as stearoyl, R₁ and R₂ are methyl or ethyl, and n is 2 or 3. Examples of such compounds include stearmidopropyl dimethylamine. Particularly preferred are amidoamine compounds complexed with a mild dimer acid, such as di(behenamidopropyl dimethyl amine) dimer dilinoleate or di(linoleamidopropyl dimethyl amine) dimer linoleate. Both ingredients are sold by Alzo, Inc. under the NECON tradename.

Also, quaternary imidazolinium salts having the following general formula are suitable as the cationic conditioning agent:

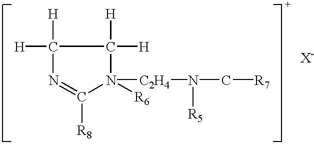

wherein R₅ is hydrogen or a C₁₋₄ alkyl; R₆ is a C₁₋₄ alkyl; R₇ is a C₈-₂₂ alkyl; and R₈ is hydrogen, or a C₁₋₂₂ alkyl; and X is an anion as defined above.

Also suitable as the cationic hair conditioning agent are salts of fatty primary, secondary, or tertiary amines, wherein the substituted groups have 12 to 22 carbon atoms. Examples of such amines include dimethyl stearamine, dimethyl soyamine, stearylamine, myristylamine, tridecylamine, ethyl stearamine, and so on.

(c) Fatty Alcohols

It may also be desirable to include one or more fatty alcohol structuring agents in the hair conditioner compositions. Suggested ranges are 0.1-20%, preferably 0.5-15%, more preferably 1-10% by weight of the total composition. Fatty alcohols as described herein in Section II.A.1(h) are suitable.

Also suitable for use in the hair conditioner compositions are ingredients mentioned in Section II.A.1, such as natural or synthetic polymers, or protein derivatives in the same ranges as taught therein.

5. Makeup Compositions

Also suitable as the vehicle are makeup composition such as foundation, blush, eyeshadow, and the like, which may be in the water-in-oil or oil-in-water emulsion form, or anhydrous.

(a) Emulsion Makeup Compositions

Suitable emulsion makeup compositions generally comprise 1-95%, preferably 5-80%, more referably 10-70% by weight of the total composition of water; and 0.1-65%, preferably 0.5-60%, more preferably 1-50% by weight of the total composition of oil; and 0.1-40%, preferably 0.5-25%, more preferably 1-20% by weight of the total composition of pigment. If desired the compositions may also contain additional ingredients such as surfactants, humectants, film forming polymers and the like.

Suitable oils include the silicones mentioned in Section II.A.1(a)(i) above, as well as the paraffinic hydrocarbons and organic oils mentioned in Section II.A.1(b)(i) and (ii) above. Suitable pigments are those mentioned in Section II.A.3(c) above. Suitable additional ingredients are those mentioned in Section II.A.1, above, in the ranges set forth therein.

(c) Anhydrous Makeup Compositions

Suitable anhydrous makeup compositions for use as the delivery vehicle include blush, anhydrous foundation, eyeshadow, lipstick and the like. Generally, such compositions comprise about 0.1-40% pigment, 0.1-50% oil, and 0.1-45% of a structuring agent such as a wax or synthetic polymer. Suitable pigments, oils, and structuring agents are as mentioned in Section II.A.1, above.

6. Mascara Compositions

Mascara compositions are also suitable cosmetic compositions for use in delivering the keritinization modulating compound to keratinous surfaces such as eyelashes. Generally such mascara compositions are in the form of water and oil emulsions and comprise about 0.1-50% water and 0.1-50% oil, in addition to 0.1-45% pigment. In addition, the mascara compositions may contain one or more synthetic polymers ranging from about 0.1-45% by weight of the total composition, as well as about 0.1-45% of one or more structuring agents, surfactants and other desirable ingredients. Suggested ranges of surfactant (or emulsifier) are 0.1-25%, preferably 0.5-20%, more preferably 1-15% by weight of the total composition. The oil, pigments, synthetic polymers, structuring agents, and surfactants, suitable for use therein are set forth in Section II.A.1, above.

The invention will be further described in connection with the following examples, which are set forth for the purpose of illustration only.

### EXAMPLE 1

A skin cream was made according to the following formula:

| | w/w % |
|---|---|
| Water | 53.47 |
| Allantoin | 0.20 |
| Glycerin | 3.00 |
| Methyl paraben | 0.25 |
| Palmitoyl hydrolyzed wheat protein | 1.00 |
| Triethanolamine | 0.15 |
| Glycerinlwater/PEG-8/caprylyl glycol/ | 5.00 |
| sodium polyacrylate | |
| Petrolatum | 2.00 |
| Squalane | 4.00 |
| Butyl methoxy dibenzoylmethane | 0.25 |
| Butylated hydroxy toluene | 0.02 |
| Propyl paraben | 0.05 |
| Octyl methoxycinnamate | 0.75 |
| Diazolidinyl urea | 0.15 |
| Sodium hyaluronate | 0.01 |
| Tocopheryl acetate | 0.30 |
| Sodium PCA | 1.00 |
| Fragrance | 0.30 |
| Cyclomethicone and dimethiconol | 2.50 |
| Behenyl alcohol/arachidyl alcohol/stearyl alcohol | 3.00 |
| Coco-caprylate/caprate | 2.00 |
| Shorea stenoptera butter | 5.00 |
| Isononyl isononanoate | 5.00 |
| Pentaerythrityl tetraoctanoate | 2.50 |
| Glyceryl behenate | 2.00 |
| Polyacrylamide/water/C13-14 isoparaffin/ | 2.00 |
| laureth-7 | |
| Biosaccharide gum-1/phenoxyethanol/parabens | 1.00 |
| Palmitoyl-methoxytryptamine | 2.00 |
| Water/propylene glycol/hydrolyzed soy protein/ | 1.00 |
| phenoxyethanol/aloe barbadensis extract/ | |
| parabens | |
| Phenoxyethanol/methyldibromo glutaronitrile | 0.10 |

The composition was made by combining the ingredients and mixing well to form a white colored cream that was stored in glass cosmetic containers.

### EXAMPLE 2

A skin treatment serum was made according to the following formula:

| | w/w % |
|---|---|
| Water | QS |
| Carbopol 10 | 0.20 |
| Glycerin | 2.00 |
| Tetrasodium EDTA | 0.02 |
| Xanthan Gum | 0.20 |
| Glucose/sorbitol/citric acid | 2.00 |
| PEG-8/SMDI copolymer | 1.00 |
| Triethanolamine | 0.20 |
| Diazolidinyl urea | 0.25 |
| PEG-8 | 5.00 |
| Styrene acrylates copolymer/hydrolyzed | 0.10 |
| collagen | |
| Fragrance | 0.36 |
| Dimethicone copolyol | 0.50 |
| Dimethicone copolyol meadowfoamate | 1.00 |
| Water/glycerin/propylene glycol/bacillus | 0.10 |
| ferment | |
| PEG-60 hydrogenated castor oil | 1.00 |
| Colorants | 0.30 |
| Palmitoyl methoxytryptamine | 3.00 |

### EXAMPLE 3

A toner composition for use in cleansing skin was made according to the following formula:

| | w/w % |
|---|---|
| PPG-2-Ceteareth-5 | 3.00 |
| Trideceth-9, PEG-5 Octanoate | 2.00 |
| Menthol | 0.01 |
| Fragrance | 0.20 |
| Water | QS |
| Sericin | 0.05 |
| Hydrolyzed sweet almont protein | 0.05 |
| Propylene glycol | 4.00 |
| Preservatives | 0.30 |
| Tetrasodium EDTA | 0.09 |
| Benzophenone 4 | 0.05 |
| Diazolidinyl urea | 0.25 |
| Colorants | 0.01 |
| Palmitoyl methoxytryptamine | 0.50 |

The composition was made by combining all of the ingredients and mixing well.

### EXAMPLE 4

A cleansing composition was made according to the following formula:

| | w/w % |
|---|---|
| Water | QS |
| Preservatives | 0.30 |
| Decyl glucoside | 2.40 |
| Disodium lauroamphodiacetate/sodium trideceth sulfate/ | 12.00 |
| hexylene glycol/isopropanol | |
| Fragrance | 0.40 |
| Polyquaternium-10 | 0.06 |
| Polyquaternium-7 | 3.00 |
| Diazolidinyl urea | 0.20 |
| Propylene glycol, primula veris | 0.10 |
| Palmitoyl methoxytryptamine | 1.00 |

The composition was made by combining the ingredients and mixing well. The composition is used for cleansing skin, hair, or nails.

### EXAMPLE 5

An aqueous-based nail enamel composition is prepared as follows:

| | w/w % |
|---|---|
| Polymer Solution* | 75.00 |
| Water | 8.00 |
| Isopropyl alcohol | 11.50 |
| Dipropylene glycol methyl ether | 3.00 |
| Propylene glycol dibenzoate | 1.50 |
| Palmitoyl methoxytryptamine | 1.00 |

*Polymer is 50% butyl methyacrylate, 20% ethyl methacrylate, 20% acetoacetoxyethylmethacrylate, and 10% acrylic acid, (all percentages by weight), neutralized with ammonium hydroxide. The Polymer Solution contains 40% w/w Polymer and 60% w/w of a solution comprised (by weight of the total solution) of 30% water, 20% isopropyl alcohol, and 10% methoxypropanol.

### EXAMPLE 6

Solvent based nail enamel compositions are made according to the following formulas:

| | w/w % | | | |
|---|---|---|---|---|
| Formula | A | B | C | D |
| Polymer 1* | 38.80 | — | — | — |
| Polymer 2* | — | 27.40 | — | 30.80 |
| Polymer 3* | — | — | 29.50 | — |
| Nitrocellulose | 2.90 | 2.80 | 2.50 | 2.80 |
| Diisopropyl adipate | 1.90 | 1.80 | 1.60 | 1.80 |
| Stearalkonium bentonite | 1.00 | 1.00 | 0.90 | 1.00 |
| Butyl acetate | 11.80 | 35.80 | 32.70 | 32.80 |
| Ethyl acetate | 39.90 | 27.50 | 29.30 | 29.10 |
| Isopropyl alcohol | 0.50 | 0.50 | 0.30 | 0.50 |
| Silicone glycol copolymer** | 0.10 | 0.10 | 0.10 | 0.10 |
| Propylene carbonate | 1.00 | 1.00 | 1.00 | 1.00 |
| Acetyl tributyl citrate | 0.15 | 0.15 | 0.15 | 0.15 |
| Titanium dioxide | 0.25 | 0.25 | 0.25 | 0.25 |
| D&C Red #7, Ca Lake | 0.05 | 0.05 | 0.05 | 0.05 |
| Red iron oxide | 0.55 | 0.55 | 0.55 | 0.55 |
| Black iron oxide | 0.10 | 0.10 | 0.10 | 0.10 |
| Palmitoyl methoxytryptamine | 1.00 | 1.00 | 1.00 | 1.00 |

*Polymer 1 comprises 80% butyl methacrylate, 20% acetoacetoxyethylmethacrylate. the polymer had a weight average molecular weight of 47,900, a number average molecular weight of 25,100, a polydispersity of 1.91 and a glass transition temperature of 8° C.
*Polymer 2 comprises 70% butyl methacrylate, 20% acetoacetoxyethylmethacrylate, and 10% acrylic acid
*Polymer 3 comprises 80% butyl methacrylate, 10% acetoacetoxyethylmethacrylate, and 10% acrylic acid
**Dow Corning 1248 fluid, dimethyl, methyl hydrogen siloxane, reaction product with polypropylene glycol monoallyl ether.

### EXAMPLE 7

A hair conditioner composition is made according to the following formula:

| | w/w % |
|---|---|
| Water | QS |
| Citric acid | 0.15 |
| Methyl paraben | 0.20 |
| Propyl paraben | 0.05 |
| Panthenol | 0.01 |
| Cetearyl alcohol | 2.00 |
| Stearyl alcohol | 1.20 |
| Cetyl alcohol | 2.00 |
| Propylene glycol | 2.00 |
| Ceteareth-20 | 0.50 |
| Trimethylsilylamodimethicone | 1.00 |
| Fragrance | 0.50 |
| Pantethine | 0.001 |
| Behentrimonium chloride | 2.70 |
| Dimethicone/dimethiconol | 0.30 |
| Dilinoleamidopropyl dimethylamine dimer linoleate | 1.40 |
| Propylene glycol dicaprylate/dicaprate/ | 2.0 |
| /PPG1-trideceth-6* | |
| Octyldodecyl fluoroheptyl citrate | 1.0 |
| and cyclomethicone (40%) | |
| Isostearyl malate/lactate/glycolate/citrate** | 0.30 |
| Palmitoyl methoxytryptamine | 1.00 |

*Polyquaternium 37
**Dermol ALFA, Alzo Inc.

The above composition was made by mixing the ingredients in water and stirring

### EXAMPLE 8

A makeup composition is made according to the following formula:

| | w/w % |
|---|---|
| Cyclomethicone | 3.00 |
| Propyl paraben/laureth-7 | 0.75 |
| Mica/methicone | 0.01 |
| Red iron oxide/methicone | 2.70 |
| Yellow iron oxide/methicone | 2.70 |
| Black iron oxide/methicone | 2.70 |
| Titanium dioxide/cyclomethicone/ | 14.10 |
| dimethicone copolyol | |
| Zinc oxide/cyclomethicone/dimethicone | 5.00 |
| copolyol | |
| Cyclomethicone/Titanium dioxide/dimethicone copolyol/ | 3.80 |
| triethoxy caprylyl silane | |
| Spherical silica | 0.15 |
| Nylon-12 | 1.00 |
| Boron nitride | 1.05 |
| Titanium dioxide/methicone | 1.00 |
| Dimethicone | 7.25 |
| Cyclomethicone | 5.80 |
| Tribehenin | 0.10 |
| Retinyl palmitate | 0.01 |
| Tocopheryl acetate | 0.01 |
| Aloe extract | 0.01 |
| Dimethicone | 1.50 |
| Polyglyceryl-4-isostearate | 1.50 |
| Cyclomethicone/dimethicone | 3.40 |
| Water | 30.05 |
| Salicylic acid/hydrolyzed vegetable protein | 0.50 |
| Methoxypropylgluconamide | 0.50 |
| Magnesium ascorbyl phosphate | 0.01 |
| Ethyl paraben/propylene glycol | 5.75 |
| Propylene glycol | 2.37 |
| Tetrasodium EDTA | 0.01 |
| Magnesium sulfate | 0.01 |
| Chamomile extract | 0.01 |
| Phytoclar | 0.01 |
| Soy protein | 3.00 |
| Cyclomethicone/dimethiconol | 2.00 |
| Methyl dihydrojasmonate | 0.25 |
| Palmitoyl methoxytryptamine | 1.00 |

The composition was prepared by combining the ingredients and mixing well to form an emulsion. The resulting makeup composition was poured into containers.

### EXAMPLE 9

A mascara composition is made according to the following formula:

| | | w/w % |
|---|---|---|
| 1 | Water | 38.05 |
| 2 | Acacia gum | 3.00 |
| 3 | Trisodium EDTA | 0.05 |
| 3 | Black iron oxide | 11.00 |
| 3 | Triethanolamine | 2.00 |
| 4 | Oleth-3-phosphate | 0.50 |
| 4 | Palmitoyl methoxytryptamine | 2.00 |
| 5 | Rice Wax | 2.00 |
| 5 | Oleth-3 | 1.80 |
| 5 | Ditrimethylolpropane tetrastearate | 5.00 |
| 5 | Polyethylene | 8.00 |
| 5 | Trioctyldodecyl citrate dilinoleate | 0.50 |
| 6 | Stearic acid | 4.30 |
| 6 | N-acyl glutamic acid diamide | 1.40 |
| 7 | Propyl paraben | 0.10 |
| 8 | Phenoxyethanol | 1.00 |
| 8 | Butylene glycol | 2.00 |
| 8 | Methyl paraben | 0.30 |
| 9 | Acrylate copolymer emulsion* | 12.00 |
| 10 | Polyurethane** | 5.00 |

Sequences 1 and 2 are pre-mixed at room temperature until the acacia gum is dispersed in the water. Sequences 1 through 4 are ground in a colloid mill until the pigments are dispersed in the mixture (approximately 10 minutes). Sequences 5 and 6 are heated on a hot plate with continuous stirring at a temperature of approximately 110° C. until the mixture becomes clear, light yellow. The mixture is allowed to cool to 85 to 90° C., and Sequence 7 is then added with mixing. When the mixture is cooled to 40 to 45° C. Sequence 8 is added. The mixture is heated to 50° C. and Sequence 9 added. When the temperature of the mixture reaches 60° C. Sequence 10 is added. The mixture is cooled to room temperature and filled into mascara containers.

### EXAMPLE 10

A lipstick composition is prepared as follows:

| | |
|---|---|
| 1 Candelilla wax | 1.00 |
| 1 Synthetic wax | 9.00 |
| 1 Paraffin | 4.00 |
| 1 Cetyl alcohol | 3.00 |
| 1 C10-30 cholesterol/lanasterol esters | 8.00 |
| 1 Palmitoyl methoxytryptamine | 2.00 |
| 2 Quaternium-18 hectorite | 0.20 |
| 3 Titanium dioxide | 0.95 |
| 3 D&C Red Calcium Lake | 0.60 |
| 3 Mica | 6.00 |
| 3 Iron oxides | 1.00 |
| 3 Trioctyl dodecyl citrate | 7.00 |
| 4 Emulsifying siloxane elastomer* | 3.00 |
| 4 Silicone/acrylate graft copolymer* | 20.00 |
| 4 Isotridecyl isononanoate | 17.125 |
| 4 Trioctyl dodecyl citrate | 17.125 |

*KSG21: Shin-Etsu Silicones of America
*KSG545: Shin-Etsu Silicones of America

Phase 5 is mixed well and heated to 70° C. Separately the Phase 3 ingredients are mixed well and ground with a Silverson homogenizer at a speed of 5000 rpm. All Phases 1-4 are combined and heated to 90° C. for 60 minutes to mix well. The compositions are poured into lipstick molds and allowed to cool.

### EXAMPLE 10

An anhydrous makeup stick is prepared as follows:

| | | w/w % |
|---|---|---|
| 1 | Glyceryl rosinate | 10.0 |
| 2 | Isododecane | 49.0 |
| 2 | Palmitoyl methoxytryptamine | 1.0 |
| 3 | Sodium stearate | 6.0 |
| 4 | Butylene glycol | 3.8 |
| 5 | Yellow iron oxide | 2.1 |
| 6 | Red iron oxide | 0.66 |
| 7 | Black iron oxide | 0.13 |
| 8 | Titanium dioxide | 8.0 |
| 9 | Cyclomethicone | 20.0 |
| 10 | Emulsifying siloxane elastomer | 19.31 |

Ingredients 5-10 are mixed and ground in a homogenzier at 5000 rpm for 10 minutes. Separately, Ingredients 1-4 are mixed well and heated to 85° C. The two phases are combined and mixed well. The composition is poured into stick molds and allowed to cool.

### EXAMPLE 11

A nail strengthening composition was made according to the following formula:

| | w/w % |
|---|---|
| Water | 60.21 |
| Citric acid | 0.05 |
| Glycerin | 5.00 |
| Magnesium ascorbyl phosphate | 0.05 |
| EDTA | 0.05 |
| Butylene glycol | 5.00 |
| Ethyl paraben | 0.20 |
| Methyl paraben | 0.30 |
| Xanthan gum | 0.25 |
| Magnesium aluminum silicate | 0.70 |
| Folic acid | 0.01 |
| Steareth-20 | 0.20 |
| Steareth-2 | 0.20 |
| Cetyl alcohol | 0.65 |
| Cyclomethicone | 12.00 |
| Glyceryl stearate | 0.25 |
| Propylene glycol dicaprylate/dicaprate | 4.00 |
| Stearyl alcohol | 0.55 |
| Tocopherol | 0.025 |
| Dimethicone | 2.50 |
| Urea | 0.30 |
| Methoxypropylgluconamide | 5.00 |
| Sodium hyaluronate/glycosaminoglycans | 0.10 |
| Methoxypropylgluconamide/glyceryl distearate | 0.50 |
| Thioctic acid | 1.50 |
| Pantetheine | 2.00 |
| Linoleamidopropyl-PG dimonium chloride | 1.00 |
| Pyridoxine | 0.10 |
| Biotin | 0.01 |
| Spermine | 0.01 |

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A method for cosmetically treating human keratinous surfaces to ameliorate the adverse effects of aging and environment thereon by modulating keratinization of superficial epithelial cells forming said keratinous surfaces, comprising contacting said keratinous surfaces with a lipophilic keratinization modulating compound in a cosmetically acceptable carrier.

2. The method of claim 1 wherein the lipophilic keratinization modulating compound is receptor inactive.

3. The method of claim 2 wherein the lipophilic keratinization modulating compound is a methoxytryptamine compound substituted with a lipophilic radical.

4. The method of claim 3 wherein the methoxytryptamine compound has the general formula: 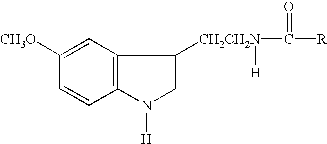
wherein R is a C11 to 19 linear or branched, saturated or unsaturated, alkyl, that may be substituted with one or more hydroxyl groups.

5. The method of claim 1 wherein the keratinous surface is skin, nails, or hair.

6. The method of claim 5 wherein the keratinous surface is skin, and the cosmetic composition is a lotion, cream, gel, or serum.

7. The method of claim 6 wherein the cosmetic composition is a water and oil emulsion cream.

8. The method of claim 7 wherein the cream additionally comprises one or more polymeric film forming agents.

9. A method for modulating keratinization in human superficial epithelial cells forming human keratinous surfaces, comprising applying to said keratinous surfaces a cosmetic composition comprising a lipophilic, keratinization modulating compound in a cosmetically acceptable carrier.

10. The method of claim 9 wherein the lipophilic keratinization modulating compound is receptor negative.

11. The method of claim 10 wherein the lipophilic keratinization modulating compound is a methoxytryptamine compound.

12. The method of claim 11 wherein the methoxytryptamine compound has the following general formula: 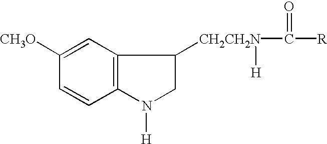
wherein R is a C11 to 19 linear or branched, saturated or unsaturated, alkyl, that may be substituted with one or more hydroxyl groups.

13. The method of claim 12 wherein the methoxytryptamine compound is palmitoyl-methoxytryptamine.

14. A cosmetic composition for application to human keratinous surfaces comprising a receptor negative, lipophilic methoxytryptamine compound in a cosmetically acceptable carrier.

15. The composition of claim 14 which is a skin lotion or cream in the water and oil emulsion form.

16. The composition of claim 15 wherein the skin lotion or cream comprises, by weight of the total composition:
0.001-30% of a lipophilic keratinization modulating compound,
1-95% water; and
10-85% oil.

17. The composition of claim 16 further comprising 0.01-65% by weight of the total composition of one more silicone polymers.

18. The composition of claim 17 wherein the silicone a nonvolatile silicone, a volatile silicone, or mixtures thereof.

19. The composition of claim 16 further comprising 0.01-15% by weight of the total composition of one or more polymers from ethylenically unsaturated monomers.

20. The composition of claim 15 which is selected from the group consisting of skin lotion, skin cream, makeup, hair conditioner, skin cleansing composition, hair cleansing composition, and mascara.
